(19) United States
(12) Patent Application Publication
(10) Pub. No.: US 2025/0058061 A1
(43) Pub. Date: Feb. 20, 2025

(54) ATOMIZATION TRIGGER STRUCTURE, AND ATOMIZER HAVING SAME

(71) Applicant: SUNSHINE LAKE PHARMA CO., LTD., Dongguan, Guangdong (CN)

(72) Inventors: Bowen CHEN, Dongguan (CN); Xuqi LIN, Dongguan (CN); Li CAO, Dongguan (CN); Zhihui FU, Dongguan (CN); Sumin HE, Dongguan (CN); Jinsong YOU, Dongguan (CN); Fangfang HUANG, Dongguan (CN)

(73) Assignee: SUNSHINE LAKE PHARMA CO., LTD., Dongguan, Guangdong (CN)

(21) Appl. No.: 18/723,815

(22) PCT Filed: Dec. 27, 2022
(86) PCT No.: PCT/CN2022/142141

§ 371 Date: Jun. 24, 2024

(30) Foreign Application Priority Data
Dec. 28, 2021 (CN) 202111632254.1

(51) Int. Cl. A61M 11/02; A61M 15/00; A61P 11/00

(52) U.S. Cl. CPC A61M 11/02; A61M 15/0001; A61P 11/00

(57) ABSTRACT

An inspiratory trigger atomizer for a patient with an airway disease, including: a main body including an atomization module and a nozzle connected thereto; an atomization driver; an air pressure sensor electrically connected to the driver, and a gas guide channel arranged in the main body, with a first end located in the nozzle and a second at a monitoring end of the sensor. The sensor acquires the air pressure in the nozzle that changes with the patient's breathing, identifies the inhalation state of the patient and generates a corresponding signal to trigger a main unit module to start the atomization driver. The atomizer has an extremely short inspiratory trigger response time, such that atomization can be quickly triggered after the patient inhales, and with atomization duration being 1.5 s, the patient can fully inhale a drug, so the atomizer can be applied to a wide range of people.

### CROSS-REFERENCE TO RELATED APPLICATION

This is a U.S. national stage application of the International Patent Application No. PCT/CN2022/142141, filed on Dec. 27, 2022, which claims the priority and benefits of Chinese Patent Application No.s 202111632254.1, filed with the State Intellectual Property Office of China on Dec. 28, 2021, which is incorporated herein by reference in their entirety.

### FIELD OF THE INVENTION

The present invention belongs to the technical field of medical devices, and specifically relates to an atomization trigger structure and an atomizer thereof.

### BACKGROUND

The spray method for the treatment of respiratory diseases is a more popular medical method in recent years, because the atomizer sprays out the atomized particles can be 1-5 μm size droplets (such as liquid medicine), which can reach the bronchi and lungs for treatment, providing inhalation of atomized particles from the mouth and nose into the bronchi, and then diffuse to the entire alveoli, so that the atomized liquid can be fully absorbed by the human body, and the effect provided by this method is more efficient and direct than that of general oral drugs.

The existing portable quantitative inhalation preparation, only the dry powder inhaler is through the patient to actively inhale the powder into the lungs, and the rest, such as spray, aerosol, are all after the patient touches the spray switch, and then cooperate with the inhalation, this way of administration has high requirements for the patient's hand-mouth coordination, and many patients make mistakes in medication, especially in some precise drug administration scenarios, will have a great impact on the accuracy of drug administration, so that the effect of treatment deviates from the target level.

### SUMMARY

The object of the present invention is to provide an atomization trigger structure and an atomizer thereof to solve the problem that the manual trigger mode in the prior art is prone to error and then affects the accuracy of the drug administration.

The atomization trigger structure provided by the invention comprises: a main body comprising an atomization module and a nozzle connected to the atomization module; an atomization driver provided on the atomization module; an air pressure sensor arranged in the atomization module and electrically connected to the atomization driver; and a gas guide channel arranged in the atomization module, and the first end of the gas guide channel is placed between a first inlet and a second outlet, the first end is located in the nozzle, and the second end is arranged at a monitoring end of the air pressure sensor; the atomization trigger structure is a net atomizer trigger structure, which solves the hand-mouth coordination problem of patients when using.

The atomization trigger structure described above is further preferably wherein the gas guide channel comprises a first channel and a second channel that are connected, the first channel is arranged in the nozzle, and the second channel is arranged in the atomization module.

The atomization trigger structure described above is further preferably wherein a trachea is arranged in the nozzle, and the inner cavity of the trachea is the first channel; the atomization module is provided with a pores, and the inner cavity of the pores is a second channel.

The atomization trigger structure described above is further preferably wherein the trachea is a hose, the first end of the hose is located in the inner cavity of the nozzle, and the second end is sealed and inserted in the pores.

The atomization trigger structure described above is further preferably wherein the nozzle further comprises: a first pipe body, the first pipe body is provided with a first inlet, a first outlet and a first guide channel connecting the first inlet and the first outlet; a second pipe body, the second pipe body is provided with a second inlet, a second outlet and a second guide channel connecting the second inlet and the second outlet; the end of the second pipe body is inserted in the first pipe body, and the second outlet is located between the first inlet and the first outlet; the trachea is threaded on the first pipe body, and the first end is located in the first guide channel, and the second end is located at the outer side of the second pipe body.

The atomization trigger structure described above is further preferably wherein the first end of the trachea is placed between the first inlet and the second outlet.

The atomization trigger structure described above is further preferably wherein the angle between the central axis of the second inlet and the second outlet is 100°˜160°; the trachea comprises a first section and a second section that are connected, and the first section and the second section are parallel to the central axis of the second inlet and the second outlet respectively.

The atomization trigger structure described above is further preferably wherein the air pressure sensor is a waterproof air pressure sensor.

The atomization trigger structure described above is further preferably wherein the response time range of described air pressure sensor is 1 ms˜1000 ms; the pressure accuracy range of the air pressure sensor is 1 Pa˜100 hPa.

The present invention also discloses an atomizer, which comprises the atomization trigger structure described in any one of the above.

The atomization trigger structure provided by the invention comprises the main body, the air pressure sensor and the gas guide channel, wherein, the main body comprises the atomization module and the nozzle connected to the atomization module, the atomization driver provided on the atomization module; the air pressure sensor is arranged in the atomization module and electrically connected to the atomization driver; and the gas guide channel is arranged in the main body, and the first end is located in the nozzle, and the second end is arranged at a monitoring end of the air pressure sensor. The above-mentioned structure realizes the automatic triggering of the automatic atomization drive by arranging an air pressure sensor that is electrically connected to the atomization driver. Specifically, the air pressure sensor can acquire the information regarding air pressure in the nozzle that changes with the patient's inhalation and exhalation via the gas guide channel, and then identify the inhalation state of the patient and generate a corresponding signal to trigger the atomization driver to realize the atomization trigger control. The above setting not only makes it more convenient for patients to take medication, but also makes the inhaled dose more controllable, so as to achieve better treatment effect.

The present invention also provides a spray assembly, comprising a spray and an aforesaid atomization trigger structure, and the active pharmaceutical ingredient in the spray is at least one of β2 receptor agonist, glucocorticoid, muscarinic receptor antagonist and phosphodiesterase 4 inhibitor. In some embodiments, the spray assembly comprises a β2 receptor agonist unilateral spray. In some embodiments, the spray assembly comprises a glucocorticoid unilateral spray. In some embodiments, the spray assembly comprises a muscarinic receptor antagonist unilateral spray. In some embodiments, the spray assembly comprises a phosphodiesterase 4 inhibitor unilateral spray. In some embodiments, the spray assembly comprises a β2 receptor agonist and a glucocorticoid combination spray. In some embodiments, the spray assembly comprises a β2 receptor agonist and a muscarinic receptor antagonist combination spray. In some embodiments, the spray assembly comprises a muscarinic receptor antagonist and a glucocorticoid combination spray. In some embodiments, the spray assembly comprises a β2 receptor agonist, a muscarinic receptor antagonist and a glucocorticoid tripartite spray.

In some embodiments, the β2 receptor agonist comprises at one least of salbutamol or pharmaceutically acceptable salts thereof, fenoterol or pharmaceutically acceptable salts thereof, terbutaline or pharmaceutically acceptable salts thereof, formoterol or pharmaceutically acceptable salts thereof, olodaterol or pharmaceutically acceptable salts thereof, arformoterol or pharmaceutically acceptable salts thereof, indacaterol or pharmaceutically acceptable salts thereof, vilanterol or pharmaceutically acceptable salts thereof. The salbutamol pharmaceutically acceptable salt can be salbutamol sulfate salt, salbutamol hydrochloride salt. The formoterol pharmacologically acceptable salt can be formoterol fumarate salt. The olodaterol pharmacologically acceptable salt can be olodaterol hydrochloride salt. The arformoterol pharmacologically acceptable salt can be arformoterol tartrate salt. The indacaterol pharmacologically acceptable salt can be indacaterol maleate salt. The vilanterol pharmacologically acceptable salt can be vilanterol triphenylacetate salt.

In some embodiments, the glucocorticoids comprise at one least of fluticasone or pharmacologically acceptable salts or esters thereof, mometasone or pharmacologically acceptable salts or esters thereof, ciclesonide or pharmacologically acceptable salts or esters thereof, beclomethasone or pharmacologically acceptable salts or esters thereof, flunisolide or pharmacologically acceptable salts or esters thereof, budesonide or pharmacologically acceptable salts or esters thereof, triamcinolone or pharmacologically acceptable salts or esters thereof, dexamethasone or pharmacologically acceptable salts or esters thereof. The fluticasone pharmacologically acceptable salt can be fluticasone furoate salt, fluticasone fropionate salt. The mometasone pharmacologically acceptable salt can be mometasone furoate salt. The beclomethasone pharmacologically acceptable salt can be beclometasone dipropionate salt, beclometasone propionate salt. The dexamethasone pharmacologically acceptable salt can be dexamethasone sodium phosphate salt.

In some embodiments, the muscarinic receptor antagonist comprises at one least of tiotropium or pharmaceutically acceptable salts thereof, glycopyrronium or pharmaceutically acceptable salts thereof, umeclidinium or pharmaceutically acceptable salts thereof, aclidinium or pharmaceutically acceptable salts thereof, ipratropium or pharmaceutically acceptable salts thereof, oxitropium or pharmaceutically acceptable salts thereof, revefenacin or pharmaceutically acceptable salts thereof. The tiotropium pharmacologically acceptable salt can be tiotropium bromide salt. The glycopyrronium pharmacologically acceptable salt can be glycopyrronium bromide salt. The umeclidinium pharmacologically acceptable salt can be umeclidinium bromide salt. The aclidinium pharmacologically acceptable salt can be umeclidinium bromide salt. The ipratropium pharmacologically acceptable salt can be ipratropium bromide salt. The oxitropium pharmacologically acceptable salt can be oxitropium bromide salt.

In some embodiments, the phosphodiesterase 4 inhibitor compreses at least one of roflumilast or pharmacologically acceptable derivatives thereof, and apremilast or pharmacologically acceptable derivatives thereof. The roflumilast pharmacologically acceptable derivatives can be roflumilast N-oxide.

The present invention also provides a spray assembly, comprising a spray and an aforesaid atomization trigger structure, and the active pharmaceutical ingredient in the spray comprises at least one of prostacyclin, treprostinil and iloprost.

The present invention also provides a spray assembly, comprising a spray and an aforesaid atomization trigger structure, and the active pharmaceutical ingredient in the spray comprises an antibiotic or an antiviral drug. The antibiotic comprises at one least of aztreonam, tobramycin, amikacin, ciprofloxacin, the antiviral drug comprises at least one of zanamivir, Laninamivir, ribavirin.

The present invention also provides a spray assembly, comprising a spray and an aforesaid atomization trigger structure, and the active pharmaceutical ingredient in the spray comprises at least one of pirfenidone, Nintedanib.

The present invention also provides a spray assembly, comprising a spray and an aforesaid atomization trigger structure, and the active pharmaceutical ingredient in the spray comprises a small molecule cytotoxic drug or a biological agent. The small molecule cytotoxic drug comprises at least one of cisplatin, Cyclophosphamide, Etoposide, vinorelbine, paclitaxel, the biological agent comprises at least one of ipilimumab, nivolumab, durvalumab.

According to one aspect of the present invention, the invention provides a method for treating COPD (chronic obstructive pulmonary disease) and/or asthma in a human in need thereof, wherein the method comprises administering to the human a spray assembly.

In some embodiments, the invention provides a method for treating pulmonary arterial hypertension in a human in need thereof, wherein the method comprises administering to the human a spray assembly.

According to one aspect of the present invention, the invention provides a method for treating lung infection in a human in need thereof, wherein the method comprises administering to the human a spray assembly.

According to one aspect of the present invention, the invention provides a method for treating idiopathic pulmonary fibrosis in a human in need thereof, wherein the method comprises administering to the human a spray assembly.

In some embodiments, the invention provides a method for treating lung cancer in a human in need thereof, wherein the method comprises administering to the human a spray assembly.

According to the other aspect of the present invention, the aforesaid spray assembly can be applied to the transpulmonary systemic administration of small molecule drugs such as levodopa, loxapine; it can also be used for systemic administration of biologics such as insulin and insulin analogues through the lungs.

### Beneficial Effects

Compared with the prior art, the present invention has at least the following beneficial effects:

The atomization trigger structure provided by the present invention realizes the automatic triggering of the automatic atomization drive by arranging an air pressure sensor that is electrically connected to the atomization driver. Specifically, the air pressure sensor can acquire the information regarding air pressure in the nozzle that changes with the patient's inhalation and exhalation via the gas guide channel, and then identify the inhalation state of the patient and generate a corresponding signal to trigger the atomization driver to realize the atomization trigger control. The above setting not only makes it more convenient for patients to take medication, but also makes the inhaled dose more controllable, so as to achieve better treatment effect.

### DESCRIPTION OF THE DRAWINGS

In order to more clearly illustrate the specific embodiments of the present invention or the technical solutions in the prior art, the drawings that need to be used in the specific embodiments or the description of the prior art will be briefly introduced below, and it is obvious that the drawings in the following description are some embodiments of the present invention, and for those skilled in the art, other drawings can also be obtained according to these drawings without paying creative labor.

FIG. 1 is a three-dimensional schematic diagram of part of the structure in the atomization trigger structure of the present invention;

FIG. 2 is the main view of FIG. 1;

FIG. 3 is a schematic diagram of the cross-sectional structure of A-A in FIG. 2;

FIG. 4 is the left view of FIG. 1.

FIG. 5 is a three-dimensional schematic diagram of atomizer described in the present invention

Description of the reference drawings:

1—trachea, 2—first pipe body, 3—second pipe body, 4—atomization module, 5—first inlet, 6—first outlet, 7—second outlet, 8—second inlet, 9—pores, 10—gas guide channel, 11—host module.

### EXAMPLES

The technical solution of the present invention will be clearly and completely described below in conjunction with the accompanying drawings, and it is obvious that the described embodiments are part of the embodiments of the present invention, not all embodiments. Based on the embodiments in the present invention, all other embodiments obtained by a person skilled in the art without making creative labor belong to the scope of protection of the present invention.

In the description of the present invention, it should be noted that the terms “center”, “up”, “down”, “left”, “right”, “vertical”, “horizontal”, “inner”, “outer”, etc., indicate the orientation or positional relationship based on the orientation or positional relationship shown in the drawing, and are only for the convenience of describing the present invention and simplify the description, and do not indicate or imply that the device or element referred to must have a specific orientation, be constructed and operated in a specific orientation, and therefore cannot be construed as a limitation on the present invention. In addition, the terms “first”, “second”, “third” are used for descriptive purposes only and should not be construed as indicating or implying relative importance.

In the description of the present invention, it should be noted that, unless otherwise expressly specified and qualified, the terms “mounted”, “connected” or “arranged” shall be understood broadly, for example, may be fixedly connected, detachable or integrally connected; it can be mechanically or electrically connected; it can be directly connected or indirectly through an intermediate medium, or it can be a communication between two components. For ordinary technicians in the field, the specific meaning of the terms in the invention can be understood in specific circumstances.

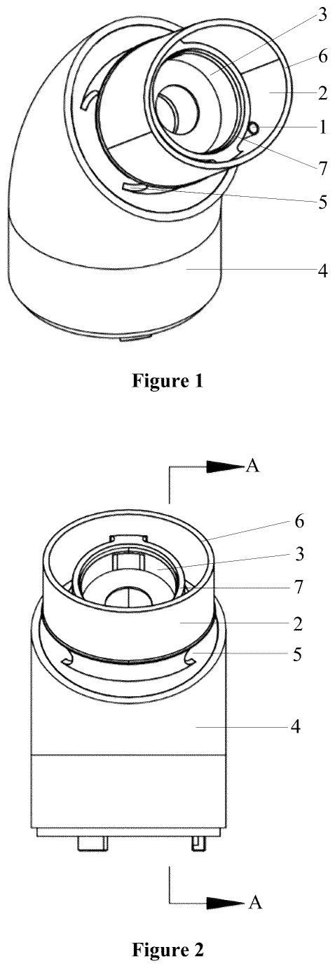

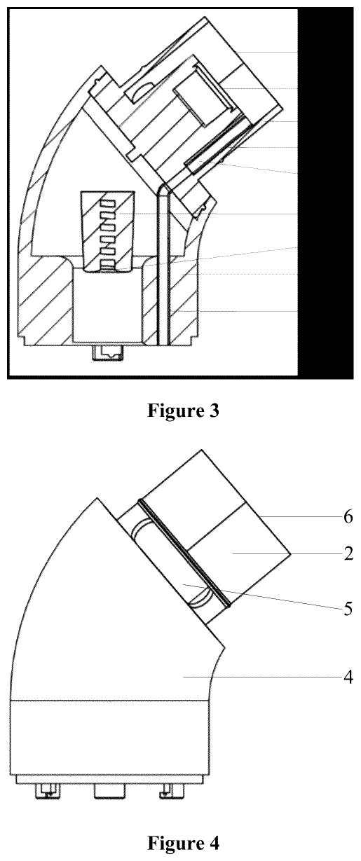

The atomization trigger structure of some embodiments of the present invention is described below with reference to FIGS. 1-4.

As shown in FIGS. 1-4, the atomization trigger structure disclosed in this embodiment mainly comprises the main body, the air pressure sensor and the gas guide channel 10.

The main body comprises the atomization module 4 (partially shown in the figure) and the nozzle, wherein the atomization module 4 is the main mounting structure, and a medicine bottle, an air pressure sensor, an atomization driver and a nozzle are arranged on it, and it is suitable for triggering the atomization driver through the air pressure sensor to drive the atomization of the medicine liquid in the medicine bottle and is sprayed out through the nozzle diversion flow. The nozzle is also used to connect with the human mouth and nose, so the air pressure in the endotracheal cavity can change according to the inhalation and exhalation of the human mouth and nose. The gas guide channel 10 is arranged in the main body, and the first end is located in the nozzle, and the second end is placed at the monitoring end of the air pressure sensor, that is, the air pressure change in the nozzle can be transmitted to the air pressure sensor through the gas guide channel 10, and then the air pressure sensor can generate a corresponding signal according to the air pressure change in the nozzle, so that the atomization driver that obtains the signal can drive the atomization of the liquid medicine according to the signal, and then realize the atomization trigger control.

The nozzle and the atomization module 4 can be an integrated structure, can also be a split structure, correspondingly, the gas guide channel 10 is arranged in a variety of ways, and the position relationship between the gas guide channel 10 and the air pressure sensor is also set in a variety of ways, and as long as the structure that can realize the above functions can be used in the present embodiment. Specifically, in this embodiment, the nozzle is detachably connected with the atomization module 4, and the gas guide channel 10 comprises the first channel and the second channel that are connected, the first channel is located in the nozzle, and the second channel is arranged in the atomization module 4.

Further, the trachea 1 is arranged in the nozzle, and the inner cavity of the trachea 1 is the first channel; the atomization module 4 is provided with a pores 9, and the inner cavity of the pores 9 is the second channel. Preferably, the trachea 1 is the hose, the first end of the trachea 1 is located in the nozzle, the second end is sealed and inserted at one end of the pores 9, and the air pressure sensor is sealed and connected with the other end of the pores 9.

In addition to the trachea 1, the nozzle further comprises the first pipe body 2 and the second pipe body 3. Specifically, the inner cavity of the first pipe body 2 is the first guide channel, one end port is the first outlet 6, the side wall of the periphery of the other end is provided with the through groove used for the first inlet 5, preferably, the first inlet 5 is plural, and the plurality of first inlets 5 are arranged on the side wall of the straight pipe in the annular circumferential interval. The first pipe body 2 is suitable for guiding external air to flow into the first guide channel from the first inlet 5 at the side wall of the first pipe body 2, and flows out into the human body mouth and nose from the first outlet 6 at the end after being guided by the first guide channel.

The second pipe body 3 is the elbow that is open at both ends, and the inner cavity of the elbow is the second guide channel, and the two ports are the second inlet 8 and the second outlet 7 respectively. The bending shape of the second pipe body 3 is suitable for adjusting the position relationship between the medicine bottle and the human oral cavity, so that when the atomizer diversion piece is in use, the medicine bottle is in an erect state, preferably, in this embodiment, the angle between the central axis of the second inlet 8 and the central axis of the second outlet 7 is 130°, and of course its angle can also be other values between 100° and 160°. The second inlet 8 is the liquid medicine inlet, specifically, the second inlet 8 is fittingly connected with the mouth of the medicine bottle, and is suitable for introducing the liquid medicine in the medicine bottle into the second guide channel. The second outlet 7 is the outlet of the atomized particle, and is provided with the atomizing sheet that is electrically connected with the atomization driver, which is suitable for atomizing the liquid medicine through the atomizing sheet, and makes the atomized particle atomized through the atomizing sheet enter the first guide channel.

The second pipe body 3 is assembled and connected through the mounting ring that is located at the end of the first pipe body 2, and specifically, the middle part of the second pipe body 3 is inserted in the central hole of the mounting ring, and the second outlet 7 is further arranged in the first guide channel and is located between the first inlet 5 and the first outlet 6. Preferably, the first pipe body 2, the second pipe body 3 and the mounting ring are integrally formed. The mounting ring is provided with a mounting hole suitable for tracheal 1 to be worn, so that the first end of trachea 1 is located in the first guide channel between the outer wall of the second pipe body 3 and the inner wall of the first pipe body 2, and the second end is located on the outside of the second guide channel. Preferably, the first end of the trachea 1 is placed between the first inlet 5 and the second outlet 7, specifically, the end face of the first end of the trachea 1 is the first interface, the plane where the edge line of the first inlet 5 near the direction of the first outlet 6 is the second interface, the plane where the second outlet 7 is located is the third interface, and the first interface is located between the second interface and the third interface. Further, the trachea 1 is bounded by the mounting ring and comprises a first section and a second section that are connected, and the first section and the second section are parallel to the central axis of the second inlet 8 and the second outlet 7 respectively, so that the angle between the first section and the second section is the same as the bending angle of the second pipe body 3, that is, the angle between the first section and the second section is 100°˜160°.

With the progress of breathing, when inhaling, negative pressure is present in the first guide channel of the nozzle, at this moment, on the one hand, the air pressure change condition is transmitted to the air pressure sensor through the trachea 1 and is obtained by it to generate a signal, the atomization driver obtains this signal and drives the atomizing sheet at the second outlet 7 to vibrate to atomize the liquid medicine, and the atomization particles generated by the vibration of the atomizing sheet enter the first guide channel; on the other hand, the external air enters the first guide channel through the first inlet 5 due to negative pressure, and because the second outlet 7 is located between the first inlet 5 and the first outlet 6, the atomized particles are located in the circulation path of the external air, and then the atomized particles entering the first guide channel can be directly coerced by the external air and brought into the human body mouth and nose. In the same way, when exhaling, there is positive pressure in the first guide channel of the nozzle, and the change in air pressure is transmitted to the air pressure sensor by trachea 1 and is obtained by it to generate a signal, and the atomization driver obtains the signal and stops driving the atomizing sheet at the second outlet 7, and the atomization of the liquid medicine stops, so as to avoid the waste of the liquid medicine. As the breath progresses, this process is repeated, so that the atomization is automatically triggered, and the inhaled dose is more controllable, resulting in a better therapeutic effect.

In addition, the nozzle through the structure of the above, on the one hand, to avoid the atomized particles stay in the first guide channel and then agglutination, improve the efficiency of drug administration, on the other hand, the external gas to promote the atomized particles forward, so that the atomized particles in the direction of the external gas forward direction, and then realize the precise control of the atomized particles airflow direction.

Further, in the atomization administration process, the atomization spray has liquid, and the inhalation and exhalation also have water vapor, so that the humidity in the nozzle is larger, and part of the water vapor can enter into the gas guide channel 10 through the trachea 1, therefore, in order to ensure the normal use of the air pressure sensor, a waterproof air pressure sensor is adopted in this embodiment. At the same time, in order to ensure the trigger sensitivity, in this embodiment, the response time range of the air pressure sensor is 1 ms˜1000 ms to meet the low time delay of the inhalation trigger; the pressure accuracy of the air pressure sensor is in the range of 1 Pa˜100 hPa to ensure that small pressure changes can be captured by it; the bearing pressure range of the air pressure sensor is 100 Pa˜100 Kpa. In addition, the air pressure sensor has a certain temperature compensation capability and is located on the circuit board away from the heat-generating device.

Further, the embodiment further discloses an atomizer that comprises the atomization trigger structure, and therefore, it has all the beneficial effects of the atomization trigger structure, and is not repeated herein.

Further, based on the atomizer described above, the following tests were also carried out.

Test 1. Simulate the absorption effect of the drug with or without delay in inhalation on the cascade impactor
1. Instruments: Anderson Cascade Impactor (ACI), Signal Generator (Specification: AFG10022, Manufacturer: Tektronix), High Voltage Amplifier (Specification: ATA-2031, Manufacturer: Antai), Vacuum Pump (Specification: LCP5, Manufacturer: Copley scientific).
2. Sample: Atomization module shown in FIG. 1.
3. Test step: the power supply of signal generator and high-voltage amplifier was turned on, the measurement frequency and amplitude required for atomizing sheet 3 were set on the signal generator, and the output voltage RMS was 25 V, and the output frequency was 101 KHz.

This product was inserted into the special nozzle adapter, connected with the timer (1.5 s), the vacuum pump was opened, the flow rate of the vacuum pump was adjusted to 28.3 L/min, test 1: the atomization module was inserted into the special nozzle adapter, the vacuum pump was turned on, sprayed once, and the gas was pumped for 5 s after the spraying, and 8 spray drugs were collected; test 2: the atomizing module was inserted into the special nozzle adapter, and the atomizing module was sprayed once. After the spraying was triggered for 1 s, the vacuum pump was turned on, and the gas was pumped for 5 s after the spraying, repeated for 8 times, and 8 spray drugs were collected.
3. The test results

Test 1

| | 8 Spray | | |
|---|---|---|---|
| | deposition | Deposition | Microparticle |
| Place | amount μg | amount % | dose % |
| Adapter | 0.03 | 0.16 | 69 |
| Suction inlet + | 0.60 | 3.14 | |
| conical opening | | | |
| Level 0 | 2.32 | 12.13 | |
| Level 1 | 3.04 | 15.89 | |
| Level 2 | 2.70 | 14.11 | |
| Level 3 | 4.44 | 23.21 | |
| Level 4 | 2.98 | 15.58 | |
| Level 5 | 1.24 | 6.48 | |
| Level 6 | 0.11 | 0.58 | |
| Level 7 | 0.18 | 0.94 | |
| Level F | 1.49 | 7.79 | |

Test 2

| | 8 Spray | | |
|---|---|---|---|
| | deposition | Deposition | Microparticle |
| Place | amount μg | amount % | dose % |
| Adapter | 0.11 | 0.95% | 37 |
| Suction inlet + | 5.07 | 43.97% | |
| conical opening | | | |
| Level 0 | 1.11 | 9.63% | |
| Level 1 | 0.97 | 8.41% | |
| Level 2 | 0.90 | 7.81% | |
| Level 3 | 1.52 | 13.18% | |
| Level 4 | 0.98 | 8.50% | |
| Level 5 | 0.38 | 3.30% | |
| Level 6 | 0.04 | 0.35% | |
| Level 7 | 0.03 | 0.26% | |
| Level F | 0.42 | 3.64% | |

Conclusion: From the comparison of the results of test 1 and test 2, test 1 started the pump before spraying (simulated inhalation trigger) than test 2 started the spray for 1 second before opening the pump and pumping (simulated existing atomizer: atomization followed by inhalation), the deposition in the adapter, suction inlet and conical opening was significantly lower, so inhalation followed by atomizer trigger had a great improvement in drug delivery efficiency (less deposition).

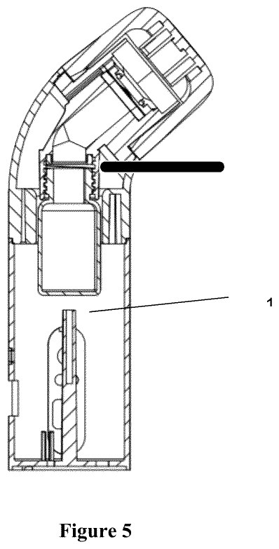

Test 2: Test of inspiratory trigger time
1. Instrument: Vacuum pump (specification: LCP5, manufacturer: Copley scientific)
2. Sample: inspiratory trigger atomizer (shown in FIG. 5)
3. Test steps: The vacuum pump was turned on, the volume flow rate of the vacuum pump was set to 15 L/min, the vacuum pump was turned off, the inhalation trigger atomizer was inserted into the special nozzle adapter, the vacuum pump was turned on, the inhalation trigger atomizer spray button was pressed, the air pressure sensor in the inhalation trigger atomizer triggered the pressure electrical signal reading, and the pressure value read by the air pressure sensor was transmitted to the computer. According to the inspiratory trigger algorithm, the starting time of the inspiratory trigger atomizer was calculated.
4. Test Results:
   Inspiratory trigger time at volumetric flow rate of 15 L/min

| Sample | Number of triggered frames | Time/s |
|---|---|---|
| 001 | 8 | 0.113 |
| 002 | 9 | 0.127 |
| 003 | 4 | 0.056 |
| 004 | 10 | 0.141 |
| 005 | 5 | 0.121 |
| 006 | 10 | 0.141 |
| 007 | 14 | 0.147 |
| Average value | 8.57 | 0.121 |

Conclusion: After multiple sample tests, the average trigger time of the inspiratory trigger nebulizer was 0.12 s, and all samples could be triggered within 0.15 s.

Test 3. Test results of atomization residue at different flow rates
1. Instruments: Anderson Cascade Impactor (ACI), Vacuum Pump (Specification: LCP5, Manufacturer: Copley scientific).
2. Sample: inspiratory trigger atomizer (shown in FIG. 5)
3. Test steps: the vacuum pump was opened, the flow rate of the vacuum pump was adjusted to 15 L/min, 28.3 L/min and 40 L/min respectively, the vacuum pump was turned off, the suction trigger atomizer was inserted into the special nozzle adapter, the vacuum pump was opened, the spray was sprayed, each spray was pumped for 5 s, and each flow rate was collected 8 sprays.
4. The test results:

| Flow rate | 15 L/min | 28.3 L/min | 40 L/min |
|---|---|---|---|
| Adapter + Suction | 1.93 | 8.99 | 7.47 |
| Inlet + conical | | | |
| opening Residual % | | | |
| Microparticle dose % | 63 | 70 | 68 |

Conclusion: Under different flow rates, the inspiratory trigger atomizer with inspiratory trigger has good drug administration efficiency (the residual ratio is <10%), which can be applied to people with different respiratory rates.

Finally, it should be noted that: the above embodiments are only used to illustrate the technical solution of the present invention, not to limit it; although the present invention has been described in detail with reference to the foregoing embodiments, a person skilled in the art should understand that it may still modify the technical solutions recorded in the foregoing embodiments, or replace some or all of the technical features therein; and these modifications or substitutions do not make the essence of the corresponding technical solutions out of the scope of the technical solutions of each embodiment of the present invention.

1. An atomization trigger structure, wherein it comprises:
a main body, comprising an atomization module and a nozzle connected to the atomization module; the atomization module is provided with atomization driver;
an air pressure sensor, the air pressure sensor is arranged in the atomization module and is electrically connected with the atomization driver;
a gas guide channel, the gas guide channel is arranged in the atomization module, and the first end of the gas guide channel is placed between a first inlet and a second outlet, the first end is located in the nozzle, and the second end is arranged at a monitoring end of the air pressure sensor;
the atomization trigger structure is a net atomizer trigger structure, which solves the hand-mouth coordination problem of patients when using.

2. The atomization trigger structure of the claim 1, wherein the gas guide channel comprises a first channel and a second channel that are connected, the first channel is arranged in the nozzle, and the second channel is arranged in the atomization module.

3. The atomization trigger structure of the claim 2, wherein a trachea is arranged in the nozzle, and the inner cavity of the trachea is the first channel; the atomization module is provided with a pores, and the inner cavity of the pores is a second channel.

4. The atomization trigger structure of the claim 3, wherein the trachea may be a hose or a hard pipe, can be integrally formed with the nozzle, and can be installed in the nozzle, the first end of the hose is located in the inner cavity of the nozzle, and the second end is sealed and inserted in the pores.

5. The atomization trigger structure of the claim 3, wherein the nozzle further comprises:
a first pipe body, the first pipe body is provided with a first inlet, a first outlet and a first guide channel connecting the first inlet and the first outlet;
a second pipe body, the second pipe body is provided with a second inlet, a second outlet and a second guide channel connecting the second inlet and the second outlet; the end of the second pipe body is inserted in the first pipe body, and the second outlet is located between the first inlet and the first outlet;
the trachea is threaded on the first pipe body, and the first end is located in the first guide channel, and the second end is located at the outer side of the second pipe body.

6. The atomization trigger structure of the claim 5, wherein the angle between the central axis of the second inlet and the second outlet is 100°˜160°; the trachea comprises a first section and a second section that are connected, and the first section and the second section are parallel to the central axis of the second inlet and the second outlet respectively.

7. The atomization trigger structure of the claim 1, wherein the air pressure sensor is a waterproof air pressure sensor.

8. The atomization trigger structure of the claim 1, wherein the response time range of the air pressure sensor is 1 ms˜1000 ms; the pressure accuracy range of the air pressure sensor is 1 Pa˜100 hPa.

9. A spray assembly, comprising a spray and the atomization trigger structure of claim 1, and the active pharmaceutical ingredient in the spray comprises at least one of β2 receptor agonist, glucocorticoid, muscarinic receptor antagonist and phosphodiesterase 4 inhibitor.

10. The spray assembly of the claim 9, wherein at least one of:
(i) the β2 receptor agonist comprises at one least of salbutamol or pharmaceutically acceptable salts thereof, fenoterol or pharmaceutically acceptable salts thereof, terbutaline or pharmaceutically acceptable salts thereof, formoterol or pharmaceutically acceptable salts thereof, olodaterol or pharmaceutically acceptable salts thereof, arformoterol or pharmaceutically acceptable salts thereof, indacaterol or pharmaceutically acceptable salts thereof, vilanterol or pharmaceutically acceptable salts thereof;
(ii) the glucocorticoids comprise at one least of fluticasone or pharmacologically acceptable salts or esters thereof, mometasone or pharmacologically acceptable salts or esters thereof, ciclesonide or pharmacologically acceptable salts or esters thereof, beclomethasone or pharmacologically acceptable salts or esters thereof, flunisolide or pharmacologically acceptable salts or esters thereof, budesonide or pharmacologically acceptable salts or esters thereof, triamcinolone or pharmacologically acceptable salts or esters thereof, dexamethasone or pharmacologically acceptable salts or esters thereof; and
(iii) the muscarinic receptor antagonist comprises at one least of tiotropium or pharmaceutically acceptable salts thereof, glycopyrronium or pharmaceutically acceptable salts thereof, umeclidinium or pharmaceutically acceptable salts thereof, aclidinium or pharmaceutically acceptable salts thereof, ipratropium or pharmaceutically acceptable salts thereof, oxitropium or pharmaceutically acceptable salts thereof, revefenacin or pharmaceutically acceptable salts thereof.

11. (canceled)

12. (canceled)

13. The spray assembly of the claim 9, the phosphodiesterase 4 inhibitor comprises at least one of roflumilast or pharmacologically acceptable derivatives thereof, and apremilast or pharmacologically acceptable derivatives thereof.

14. A method for treating COPD (chronic obstructive pulmonary disease) and/or asthma in a human in need thereof, wherein the method comprises administering to the human a spray assembly of claim 9.

15. A spray assembly, comprising a spray and the atomization trigger structure of claim 1, the active pharmaceutical ingredient in the spray comprises at least one of prostacyclin, treprostinil and iloprost.

16. A method for treating pulmonary arterial hypertension in a human in need thereof, wherein the method comprises administering to the human a spray assembly of claim 15.

17. A spray assembly, comprising a spray and the atomization trigger structure of claim 1, wherein the active pharmaceutical ingredient in the spray comprises an antibiotic or an antiviral drug, the antibiotic comprises at one least of aztreonam, tobramycin, amikacin, ciprofloxacin, the antiviral drug comprises at least one of zanamivir, Laninamivir, ribavirin.

18. A method for treating lung infection in a human in need thereof, wherein the method comprises administering to the human a spray assembly of claim 17.

19. A spray assembly, comprising a spray and the atomization trigger structure of claim 1, the active pharmaceutical ingredient in the spray comprises at least one of pirfenidone, Nintedanib.

20. A method for treating idiopathic pulmonary fibrosis in a human in need thereof, wherein the method comprises administering to the human a spray assembly of claim 19.

21. A spray assembly, comprising a spray and the atomization trigger structure of claim 1, and the active pharmaceutical ingredient in the spray comprises a small molecule cytotoxic drug or a biological agent.

22. A method for treating lung cancer in a human in need thereof, wherein the method comprises administering to the human a spray assembly of claim 21.
